(19) United States
(12) Patent Application Publication
(10) Pub. No.: US 2024/0000030 A1
(43) Pub. Date: Jan. 4, 2024

(54) Selection Methods

(71) Applicant: Agriculture Victoria Services PTY LTD, Bundoora, Victoria (AU)

(72) Inventors: Abdulqader Jighly, Heidelberg Heights (AU); Matthew James Hayden, Templestowe (AU); Hans Dieter Daetwyler, Yallambie (AU)

(73) Assignee: Agriculture Victoria Services PTY LTD, Bundoora, Victoria (AU)

(21) Appl. No.: 18/039,356

(22) PCT Filed: Dec. 17, 2021
(86) PCT No.: PCT/AU2021/051511

§ 371 Date: May 30, 2023

(30) Foreign Application Priority Data
Dec. 21, 2020 (AU) 2020904770

(51) Int. Cl. A01H 1/04; G06Q 50/02

(52) U.S. Cl. CPC A01H 1/04; G06Q 50/02

(57) ABSTRACT

The present invention relates to a method for determining phenotypic genomic estimated breeding values (pGEBVs), wherein the method comprises the steps of obtaining genetic, phenotypic, and environmental data for a population of organism genotypes; dividing the data into a reference population and a validation population; and analysing the data obtained. The present invention also relates to a method for selecting a genotype for producing an improved organism in a given environment, as well as a method for producing an improved organism.

### FIELD OF THE INVENTION

The present invention relates to methods for determining phenotypic genomic estimated breeding values. The present invention also relates to a method for selecting a genotype for producing an improved plant in a selected environment, as well as a method for producing an improved organism.

### BACKGROUND OF THE INVENTION

Efficient and consistent crop production is a world-wide challenge. The field of terrestrial agriculture is relied upon to produce vast supplies of the world's food and medicinal products and textiles. Management of the economics, logistics and sheer scale of agricultural output is a considerable undertaking. However, the world's human and animal population continues to grow and therewith demand for agricultural products, against the constant challenges faced by farmers in the production itself. These challenges include for example the inherent susceptibility of crops to climatic conditions, and many other abiotic and biotic stresses, such as invertebrate pests and microbe and viral crop infections. While there is no one solution to all of these issues, there are significant gains to be achieved from improvements in any one area, one of which in particular is the susceptibility of crops to climatic conditions.

Neither of genetic modification nor migrating a genotype or cultivar from one location to another will necessarily result in an improved crop or even one which gives suitable economic production, let alone consistently from season to season. The performance of a crop is a result of the interaction of its genotypes with the environment at that location. Certain genotypes will interact with a given environment differently and one may out-perform or under-perform as compared to another, but rarely is there a genotype that performs equally well in all environments. It is a goal though, of plant breeders, to develop high-yielding cultivars with low genotype×environment interaction (GEI) in the hopes of achieving stable cultivar performance across environments.

Traditional attempts to identify high-yielding cultivars across environments are simply through trial and error. It is immediately apparent that this is an extremely long, laborious and inefficient process; it involves planting crops comprised of different genotypes in different locations and diligently monitoring the performance indicators and environmental conditions year upon year.

As such, there are significant potential advantages to be gained from circumventing this process and identifying economically productive—if not the most likely productive—combinations. Even reliable calculations that can be rapidly obtained in comparison, say to just exclude those combinations of poorest performance, would still give a significant advance.

One method which assists includes the visualisation of multi-environment trials by using a GGE biplot (Yan 2000). In this analysis, the environment main effect is removed, while the genotype main effect, as well as the GEI effect, are integrated after singular value decomposition analysis (Yan and Kang 2002). The first two or three principal components (PC) of the GGE analysis, which explain the largest proportion of the genotype plus GEI variations, are usually plotted with the environmental coordinates in a single biplot. Such biplots can be useful to infer the stability of different genotypes and to inform plant breeders about superior cultivars for different mega-environments (Yan 2001).

However, this method has its limitations. Since GGE biplots depend only on two or three PCs, a considerable proportion of the genotype and GEI variation is ignored (Gauch et al. 2008). This issue is especially critical for datasets with many heterogeneous environments in which the first few PCs only explain a small proportion of the total variation (Yang et al. 2009). These PCs can also be biased if a specific mega-environment is under-represented in the dataset. Moreover, because GGE biplots utilise only phenotypic data, new crosses cannot be compared to previous biplots, and genomic components affecting traits cannot be elucidated.

Other calculation methods have been developed in recent times to this end. For example, standard genomic best linear unbiased prediction (GBLUP) uses genomic relationships to estimate the genetic merit of an individual based on a genomic relationship matrix estimated from DNA markers. The matrix defines covariance between individuals based on observed similarity at the genomic level, though it is used mostly in livestock production. Attempts to impart genomic selection (GS) statistical models on plant calculative methods have also been made. Current GS statistical models exploit genetic correlation among different environments to model GEI and to produce more accurate genomic estimated breeding values (GEBVs) (Burgueño et al. 2012; López-Cruz et al. 2015; Cuevas et al. 2016, 2017); other models consider environmental covariates to improve the calculation accuracy for multi environmental trials (Jarquin et al. 2014; He et al. 2019). However, there remain drawbacks associated with these methods, primarily that they are inaccurate, but also computationally inefficient, they also typically require positively correlated environments for best implementation and are unable to calculate new environments not included in the reference population.

There exists a need to overcome, or at least alleviate, one or more of the difficulties or deficiencies associated with the prior art.

### SUMMARY OF THE INVENTION

In one aspect of the present invention there is provided a method for determining phenotypic genomic estimated breeding values (pGEBVs), wherein the method comprise the steps of:
a) obtaining genetic, phenotypic, and environmental data for a population of organism genotypes;
b) dividing the data of step a) into a reference population and a validation population; and
c) analysing the data obtained,
   wherein the analysis includes:
i. calculating the genotype plus genotype×environment (GGE) principle component (PC) for the data of the reference population;
ii. identifying polymorphisms in the genetic data of the reference population and calculating the polymorphism effect for each PC;
iii. calculating a genomic estimated breeding value (GEBV) for each genotype of the validation population using the calculated polymorphism effect for each PC; and
iv. converting each GEBV into a phenotypic GEBV (pGEBV) by multiplying the GEBV with an inverse of a rotation matrix, wherein the rotation matrix is (e×e), and wherein e is the number of environments in the validation population.

By “genetic data” is meant information relating to the DNA and/or RNA nucleotide sequence of an organism, preferably DNA. Preferably, the genetic data includes information relating at least to one or more taxonomic markers, being a region of DNA which allows for genetic distinction of genotypes by the presence of polymorphisms when two or more are compared.

Preferably, the genetic data includes a whole or substantially whole genome, which may be at least about 80%, 85%, 90%, 95%, 98% 99% or 100% of a complete DNA sequence.

By “environment” is meant as a set of conditions in which an organism may live. For example, in the context of a plant or animal, it may include climate (e.g. air quality, humidity, temperature, wind), soil, geographic location, light exposure, feed availability, water availability, biotic (e.g. pests and diseases which may be insects and pathogen infection) and abiotic stress (e.g. water or nutrient deficit) conditions as appropriate, that may impact on plant or animal behaviour. By “environmental data” is meant information relating to the environment in which an organism lives. The data may be qualitative and/or quantitative. In a preferred embodiment, in the context of a plant, the environmental data includes at least watering conditions, in terms of amount and/or means, e.g. rainwater or irrigated.

By a “phenotype” is meant an observable characteristic generally resulting from the interaction of a genotype and an environment. A phenotype encompasses a “trait” which refers to an associated underlying physiological or biochemical characteristic. A phenotype of a genotype may be as distinguishable from that of another genotype. By “phenotypic data” is meant information relating to one or more phenotypes of a genotype. The data may be qualitative and/or quantitative. In preferred embodiments, the phenotypic data, in the context of a plant, relates to a growth condition, and preferably yield.

By a “genotype” is meant a putative identifier assigned to an organism within a species to distinguish it from others of that species. Genotypes are often assigned based on an analysis of the genetic makeup of an organism, and generally in terms of that genetic makeup being capable of contributing to the expression of a phenotype which may be distinguishable, and/or based on a breeding or other genetic manipulation method upon observation of a distinguishable phenotype, as compared to others. For clarity, a genotype encompasses a haplotype which is an identifier assigned to an organism based on the makeup of a heritable genetic subregion (e.g. a gene, loci, group thereof); again that is generally capable of giving rise to the expression of a phenotype which may be distinguishable as compared to others. In the context of a plant, “cultivar” is synonymous with “variety” and is a plant or collection thereof comprising a single genotype or a group of selected genotypes.

By a “population of organism genotypes” is meant a number sufficient to allow their comparison in the method described herein. The population will generally be of a size to allow statistically meaningful analyses and may be tens to many hundreds or many thousands, generally limited in size by the obtainable genetic, phenotypic, and environmental data. In preferred embodiments, the population is at least 200 genotypes. Also in preferred embodiments, the population is across a mega-environment; the genetic, phenotypic, and environmental data obtained for the population of genotypes is from a mega-environment. By a “mega-environment” is generally meant a cluster of geographical regions that have a reasonably homogenous environment in which most genotypes behave similarly across regions. In a preferred embodiment, a mega-environment may be at least two geographical regions. Also in preferred embodiments, the population is across a plurality of mega-environments; the genetic, phenotypic, and environmental data obtained for the population of genotypes is from more than one mega-environment, preferably wherein the mega-environments differ in their conditions.

By a “reference population” is a subset of the population of organism genotypes. The reference population, or more accurately the genetic, phenotypic, and environmental data thereof, may be used as a reference against which the data for the validation population may be analysed. In preferred embodiments, the reference population may be of at least 100 genotypes. By a “validation population” is a different subset of the population of organism genotypes. The validation population, or more accurately the genetic, phenotypic, and environmental data thereof, may be used in comparison with the data of the reference population to extract information, for example, on certain features, characteristics or trends of the data. In preferred embodiments, the validation population may be at least 100 genotypes. To be clear, the step of obtaining genetic, phenotypic, and environmental data for a population of organism genotypes and then dividing it into a reference population and a validation population encompasses separately obtaining data for each of a reference population and a validation population. The step of dividing the data is simply to be taken to mean that the data for the reference and validation populations, however obtained, relates to the same type of data and is suitably comparable. For example. it may include the same type of genetic, phenotypic, and environmental data obtained for genotypes with the same organism species.

A genotype plus genotype×environment (GGE) analysis assesses genotype by environment interactions (GED of two-way data, GEI being a change in a phenotype of two or more genotypes measured in two or more environments. The Principal Component (PC) is determined by singular value decomposition. Mathematically, GGE is the genotype by environment data matrix after the environment means have been removed. Certain methods for calculating the GGE PC are known to those skilled in the art.

In preferred embodiments, the GGE PC is calculated using a non-linear iterative partial least squares method, preferably based on Equation 1 as follows

$${Φ}_{i j} = \frac{{\overline{y}}_{i j} - {μ}_{j}}{{S}_{j}} = {Σ}_{k = 1}^{e} {λ}_{k} {α}_{i k} {γ}_{j k} + {\overline{ε}}_{ij} \qquad ( Equation 1 )$$

where Φᵢⱼ is the genotype×environment two-way matrix of GGE effects; i is the range between 1 and g (total number of genotypes); j is the range between 1 and e (total number of environments); y̅ᵢⱼ is the best linear unbiased estimate (BLUE) of genotype i in environment j; μⱼ and Sⱼ are general mean and standard deviations for environment j respectively; λₖ is the singular value of the PC k; αᵢₖ is the eigenvector for PC k of genotype i; γⱼₖ is the eigenvector for PC k of environment k; and ϵ̅ᵢⱼ is the residual of the model associated with genotype i in environment k.

By a “polymorphism” is meant a genetic variation present at one or more positions of a nucleotide sequence which allows for genetic distinction when two or more are compared. Polymorphisms may be present on coding or non coding regions, as well as regulatory or non-regulatory regions, of the nucleotide sequence. A polymorphism may be for example an insertion, deletion, substitution, or combination thereof. In preferred embodiments, the polymorphism is at least one, if not several, single nucleotide polymorphisms (SNP). An SNP is a variation in a single nucleotide. Methods for identifying polymorphisms including SNPs are known to those skilled in the art.

The step of calculating the polymorphism effect for each GGE PC essentially determines the weight of the identified polymorphisms; the likelihood of the polymorphisms contributing to the GGE PC. Methods for calculating the polymorphism effect are known to those skilled in the art. A representative method utilises the Bayesian Ridge Regression (BRR) model.

By a “genomic estimated breeding value” (GEBV) is meant the measurable extent to which a genotype influences the expression of a phenotype. Calculating a genomic estimated breeding value (GEBV) for each genotype of the validation population using the calculated polymorphism effect for each PC essentially determines to what extent the identified polymorphisms influence the expression of a phenotype. In preferred embodiments, the GEBV is a G matrix (n×e), wherein n is the number of validation genotypes and e is the number of environments. Preferably, calculating a GEBV is based on Equation 2 as follows:

GEBV=Z\hat{β} (Equation 2)

where Z is the SNP allelic dosage matrix for the validation population and \hat{β} is the calculated polymorphism effect for each GGE PC.

By a “phenotypic genomic estimated breeding value” (pGEBV) is meant the measurable extent to which environment influences the GEBV; or in other words it relates environment to phenotype. In preferred embodiments, each pGEBV is calculated based on Equation 3 as follows:

pGEBV=G×R⁻¹ (Equation 3)

where G is an (n×e) matrix of GEBVs for the GGE PCs scaled by multiplying each PC with its standard deviation; n is the number of validation genotypes; e is the number of environments; and R⁻¹ is an inverse of the rotation matrix (e×e), or the environment coordinate matrix scaled by dividing each column on the standard deviation of the correspondence PC.

In context of a method for determining pGEBVs, by an “organism” is meant a living being, whether an animal, plant, single-celled organism or other. In context of producing or obtaining an improved organism as described herein, by an “organism” is meant the same, except that its reference to an animal does not include a human being. That is, the present invention is not intended to relate to biological processes for the generation of a human being. In preferred embodiments, the organism is an animal other than a human being, or a plant.

In the context of a plant, the plant may be any cultivable plant. In preferred embodiments, the plant is a crop plant which can be cultivated and harvested for food, animal feed, fibre, oil, any other material or industrial use. For example, the plant may be for the production of pomes, citrus, and other fruits, nuts, cereals, legumes, vegetables, herbs, spices and commodities including oil. This may include, for example, plants belonging to the genus Triticum, including T. aestivum (wheat), Hordeum, including H. vulgare (barley), Zea, including Z. mays (maize or corn), Oryza, including O. sativa (rice), Saccharum including S. officinarum (sugarcane), Sorghum including S. bicolor (sorghum), Panicum, including P. virgatum (switchgrass), Helianthus (sunflower), Brassica (canola), Vigna, Cicer, Lens, Pisum (beans) Coffea (coffee) Miscanthus, Paspalum, Pennisetum, Poa, Eragrostis, Agrostis, Brachiaria, Lolium and Festucae (grasses).

In the context of an animal, the animal may be any productive animal. In preferred embodiments, the animal is one to which practices of animal husbandry are applicable for the production of food, animal feed, fibre, or any other material or for industrial use. For example, the animal may be for the production of meat and meat-derived products, poultry, eggs, dairy, fish, wool and leather. This may include, for example, animals belonging to the genus Bos (cattle), Equus (horse), Ovis (sheep), Sus (pig), Capra (goat) and Gallus (chicken).

This method provides the advantage of a significantly more accurate method for calculating in which environment a genotype excels. For example, as determined against the reference population of the obtained data, the method calculates with much greater accuracy in which environment the genotypes of the obtained data were more productively yielding, than the two sub-models (termed GE and GxE) of the standard GBLUP model with the following Equation A:

y=μ+E+g+gE+ϵ

where y is the phenotype; μ is the intercept; E is the environmental effect E˜N(0,V_{E}σ_{E}²), V_{E}=Z_{E}Z′_{E}, Z_{E} is the incidence matrix allocating genotypes to environments; g is the genotypic effects g˜N(0,V_{g}σ_{g}²), V_{g}=Z_{g}GZ′_{g}, Z_{g} is the incidence matrix allocating phenotypes to genotypes, G is the genomic relatedness matrix estimated following the first method described in VanRaden (2008); and ϵ is the residual ϵ˜N(0, σ_{E}²). There are two sub-models; with and without GEI. gE represents the GEI effect and was equal to 0 for the model without GEI (named GE). For the model that fitted GEI (named GxE), gE˜N(0,V_{gE}σ_{gE}²), V_{gE}=V_{g}⊙V_{E}, ⊙ is the Hadamard or cell-to-cell product.

The accuracy advantage arises from the calculations that assume that all variation attributed to genotypes and GEI is captured by all PCs of the GGE analysis. For this reason, the GS on these PCs (instead of the actual phenotypes) is applied before converting the GEBVs of the PCs back to the original phenotypes.

In another advantage, the method finds particular utility in selecting a genotype for a given environment. That is, it can calculate for example, phenotype potential of genotypes in unobserved environments with improved accuracy. In the context of a plant, this may include selecting a plant genotype based on its pGEBV for cultivation in a new environment.

In preferred embodiments, the method is used for selecting a genotype for producing an improved organism in a given environment, by one or more of the following:
a) identifying a genotype with the pGEBV which correlates highest with the given environment;
b) clustering the reference environments into mega environments and then calculating multiple averages of pGEBVs per genotype for each mega environment, and identifying a genotype from the average pGEBV that correlates highest with the mega environment which best matches the given environment; and
c) identifying a genotype from the following steps:
   i. calculating a singular value decomposition for a symmetric pairwise correlation matrix (U matrix) between environments including the reference environments and the selected environment (e+1×e+1);
   ii. calculating a correlation between the U matrix, obtained in step (i), and the rotation matrix (e×e);
   iii. reordering columns of the U matrix to match an order of the rotation matrix of step (ii), reversing the sign of negative correlations; and
   iv. applying Equation 3 as follows:

pGEBV=G×R⁻¹

using the reordered U matrix instead of the R matrix, where G is an (n×e) matrix of GEBVs for the GGE PCs scaled by multiplying each PC with its standard deviation; n is the number of validation individuals; and e is the number of environments; and adding a column of zeros to the end of the G matrix to match its dimensions; and
d) based thereon, selecting an identified genotype.

Accordingly, in another aspect of the present invention, there is provided a method for selecting a genotype for producing an improved organism in a given environment, wherein the method may comprise the steps of:
(a) performing a method for determining phenotypic genomic estimated breeding values (pGEBV) as described herein; and performing one or more of:
   i. identifying a genotype with a pGEBV which correlates highest with the given environment;
   ii. clustering the reference environments into mega environments and then calculating multiple averages of pGEBVs per genotype for each mega environment, and identifying a genotype from the average pGEBV that correlates highest with the mega environment which best matches the given environment; and
   iii. identifying a genotype from the following steps:
      1. calculating a singular value decomposition for a symmetric pairwise correlation matrix (U matrix) between environments including the reference environments and the given environment (e+1×e+1);
      2. calculating a correlation between the U matrix obtained in step 1, and the rotation matrix (e×e);
      3. reordering columns of the U matrix to match an order of the rotation matrix of step 2, reversing the sign of negative correlations, and applying Equation 3 as follows:

pGEBV=G×R⁻¹
using the reordered U matrix instead of the R matrix, where G is an (n×e) matrix of GEBVs for the GGE PCs scaled by multiplying each PC with its standard deviation; n is the number of validation individuals; and e is the number of environments; and adding a column of zeros to the end of the G matrix to match its dimensions; and
(b) based thereon, selecting an identified genotype.

Preferably, when reordering columns of the U matrix to match an order of the rotation matrix, the column of the U matrix that has the highest absolute correlation coefficient value is ordered with the first column in the rotation matrix. In this process, the extra column in the U matrix that does not have high correlation with any column in the rotation matrix would become the last column in the U matrix.

In preferred embodiments, the given environment is a new environment that is not included in the reference population.

The third method is particularly accurate and accordingly advantageous. It assumes that the pGEBVs for the unobserved environment can be calculated from its correlation with the reference environments, as well as the GEBVs of the GGE principal components for the reference environments. This method showed the highest average accuracy for calculating new environments. It can also be applied in breeding programs where massive populations get screened in multiple environments or seasons with high-throughput phenotyping techniques. Moreover, this method is computationally more efficient in terms of memory and time requirements.

In preferred embodiments of selecting a genotype, an organism of the selected genotype is located to the given environment, and an improved organism is produced. In context, an “improved organism” encompasses a single organism and also a plurality. The improved organism may have any advantageous phenotype, generally as compared to an organism with a lower pGEBV or GEI correlation for the given environment. For example, in the context of a plant, preferably a plant of the selected genotype is planted in the given environment, and an improved plant is produced. In context, an “improved plant” encompasses a single plant and also a cultivar or crop. The improvement may be for example by way of a larger yield which may be characterised by a larger, denser or otherwise higher producing plant or plant part.

Accordingly, in another aspect of the present invention, there is provided a method for producing an improved organism, comprising the steps of:
(a) performing a method for determining phenotypic genomic estimated breeding values (pGEBV) as described herein;
(b) performing a method as herein described for selecting a genotype for producing an improved organism in a given environment; and
(c) locating the organism comprising said selected genotype in said given environment.

Of course, by locating an organism in a given environment encompasses tending to it; for example in the context of a plant, planting, cultivating, cropping, fertilising etc. as appropriate. In preferred embodiments of this aspect of the present invention, the method is for obtaining an improved plant.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

FIG. 1 shows the clustering and correlation coefficients of the 20 environments used in the study, as described below. Positive numbers represent positive correlations while negative numbers represent negative correlations. The two main clusters are highlighted on the upper dendrogram. FIG. 1: reference complete figure; FIG. 1A: section A of FIG. 1; FIG. 1C: section C of FIG. 1; FIG. 1B: section B of FIG. 1; FIG. 1D: section D of FIG. 1. KEY: Bo: Bozeman; Ot: Othello; Hu: Huntley; Sa: Saskatoon; Da: Davis; Im: Imperial; Ob: Obregon; RF: rainfed; and IRR: irrigation.

FIG. 2 shows a GGE biplot of 20 environments and 144 genotypes used as a reference for different genomic selection models.

FIG. 3 shows the correlation between the pGEBVs produced using 3GS and G×E models for different environments. The first two rows represent the environments of cluster 1 in FIG. 1 while the last two rows represent the environments of cluster 2.

### DETAILED DESCRIPTION OF THE EMBODIMENTS

### Outline

A computationally efficient model has been developed that combines GGE analysis with genomic selection, named 3GS, to improve the accuracy for calculating GEI. The model first estimates marker effects for all PCs produced by a GGE analysis, before using the effects to calculate GEBVs for new genotypes. Then it converts the GEBVs to pGEBV by multiplying them with the inverse of the rotation matrix.

The performance of 3GS was compared to standard GBLUP, with and without modeling GEI, using wheat grain yield data phenotypes in 20 diverse environments. Environments were grouped in two major clusters with pairwise phenotypic correlation coefficients ranging from −0.28 to 0.77. On average, 3GS showed 12% higher accuracy compared to the best GBLUP model over all environments. The accuracy advantage happens primarily in one cluster when low to negative correlations are present between environments with around 31% higher accuracy than GBLUP. A statistical method was also developed to calculate unobserved genotypes in unobserved environments with good accuracy based on their correlations with the reference environments. When run as a multithread version, the 3GS model is about 80 times faster than the GBLUP model implemented in the BGLR package (required 30 seconds vs 40 minutes for BGLR). This computational efficiency is expected to further increase for larger datasets. The 3GS model improves calculation accuracy for traits with complex GEI and exhibited enhanced performance for negatively correlated environments.

### Materials and Methods

### Testing Data

The phenotypic and genotypic data for a total of 367 spring wheat genotypes were downloaded from the TCAP database (https://triticeaetoolbox.org/wheat). The phenotypic data included grain yield records for 20 field trials conducted between 2011 and 2014 with irrigation and rain-fed treatments. The trials were distributed in seven geographical locations across the United States (Davis, Imperial, Bozeman, Huntley and Othello), Mexico (Obregon) and Canada (Saskatoon) with at least 250 genotypes per trial. Trial names were coded with the first two letters of the location name followed by the season (11 to 14) followed by the treatment (IRR for irrigation and RF for rainfed). A total of 144 genotypes with phenotypic records in almost all trials (missing rate of phenotypic records=0.8%) were used as a reference population. The remaining genotypes were used for validation to avoid overlap between the reference and validation populations. The population was genotyped with 90K Infinium single nucleotide polymorphism (SNP) chip which resulted in 22,214 SNPs after filtering for a minor allele frequency <5% and call rate <10%. Narrow sense heritability was estimated using the genomic-relatedness-based restricted maximum likelihood (GREML) analysis by fitting the genomic-relatedness matrix in the mixed linear model implemented in MTG2 software (Lee et al., 2012; Lee and van der Werf, 2016).

### GGE Biplot Analysis

GGE analysis was conducted with the nonlinear iterative partial least squares (NIPALS) method implemented in the R package ‘GGE’ (http://kwstat.github.io/ggen. The general equation for the GGE model following Yan (2000) is:

$${Φ}_{i j} = \frac{{\overline{y}}_{i j} - {μ}_{j}}{{S}_{j}} = {Σ}_{k = 1}^{e} {λ}_{k} {α}_{i k} {γ}_{j k} + {\overline{ε}}_{ij} \qquad ( Equation [ 1 ] )$$

where Φᵢⱼ is the genotype x environment two-way matrix of GGE effects; i is the range between 1 and g (total number of genotypes); j is the range between 1 and e (total number of environments); y̅ᵢⱼ is the best linear unbiased estimate (BLUE) of genotype i in environment j; μⱼ and Sⱼ are the general mean and standard deviation for environment j respectively; λₖ is the singular value of the PC k; αᵢₖ is the eigenvector for PC k of genotype i; γⱼₖ is the eigenvector for PC k of environment k; and ϵ̅ᵢⱼ is the residual of the model associated with genotype i in environment k.

### GGE+GS (3GS) Model

The 3GS model implements the following major steps:

1—Calculate the GGE PCs for the phenotypic data of the reference population (144 genotypes) using equation [1];

2—Estimate the SNP effects for each PC. The Bayesian Ridge Regression (BRR) model 25 was used to calculate SNP effects as implemented in the R package BGLR (Pérez and de Los Campos, 2014). The analysis was run with 10,000 iterations with the first 5,000 iterations considered as burn-in. The analysis was multithreaded by running each PC on a different core;

3—Calculate GEBVs for the validation population (n=223 genotypes) using the SNP effects for the PCs as GEBV=Z\hat{β} (Equation 2) where Z is the SNP allelic dosage matrix for the validation population and \hat{β} is the SNP effects estimated in Step 2; and

4—Covert the GEBVs of the GGE PCs into pGEBV for each environment with the following equation:

pGEBV=G×R⁻¹ (Equation [3])

where G is an (n×e) matrix of GEBVs for the GGE PCs scaled by multiplying each PC with its standard deviation; n is the number of validation individuals; R⁻¹ is the inverse of the rotation matrix (e×e), or the environment coordinate matrix which was scaled by dividing each column on the standard deviation of the correspondence PC. pGEBV is an (n×e) matrix so each environment had its own pGEBV values. Accuracy of genomic calculation was calculated as the Pearson correlation between pGEBV and the actual phenotypic record for each environment. To calculate standard deviations for accuracy estimations, accuracies were calculated on 100 replicates of randomly selected 80% of the validation population. Only scenarios of calculating untested genotypes in observed or unobserved (new) environments were considered for validation.

### Calculating New Environments

The GE, GxE and 3GS analyses were repeated 20 times after excluding one environment in each run to be used for validation and to assess the capability of these models to calculate new environments that were not included in the reference. The GE model resulted in a single GEBV per individual over all environments, while the GxE and 3GS models produced environment specific GEBVs for each reference environment. The following three approaches to calculate new environments were compared, of which the first two are also applicable for the GxE model:
1—Calculate the new environment using the pGEBVs of the reference environment that has the highest correlation with the target new environment;
2—Cluster the reference environments into mega environments and then calculate multiple averages of pGEBVs per genotype per each mega environment. New environments are then calculated from the average pGEBV that represents the mega environment classification of the new environment; and
3—Method specific to the 3GS model, implementing the following steps:
   a. Calculate the singular value decomposition for the symmetric pairwise correlation matrix (U matrix) between environments (e+1×e+1), including the new target environment. For missing correlation coefficients, the principal component analysis using the NIPALS algorithm was used to approximate the missing values;
   b. Calculate the correlation between the U or the rotation matrix (e+1×e+1) obtained in (a) and the rotation matrix (e×e) of the GGE analysis;
   c. Reorder the columns of U to match the order of the rotation matrix, i.e. the column of U that has the highest absolute correlation coefficient value with the first column in the rotation matrix should come first and so on. Reverse the sign of the column in U if the correlation was negative. In this process, the extra column in U that does not have high correlation with any column in the rotation matrix would become the last one in U; and
   d. Apply equation [3] by using the reordered U matrix instead of the R matrix and adding an extra column of zeros to the end of the G matrix to match the dimensions. The number of columns in pGEBV should then be e+1 columns instead of e columns.

### Genomic Best Linear Unbiased Prediction (GBLUP)

The 3GS model was compared to the standard GBLUP model with the following equation:

y=μ+E+g+gE+ϵ (Equation [A])

where y is the phenotype; μ is the intercept; E is the environmental effect E˜N(0,V_{E}σhd E²), V_{E}=Z_{E}Z′_{E}, Z_{E} is the incidence matrix allocating genotypes to environments; g is the genotypic effects g˜N(0,V_{g}σ_{g}²), V_{g}GZ′_{g}, Z_{G} is the incidence matrix allocating phenotypes to genotypes, G is the genomic relatedness matrix estimated following the first method described in VanRaden (2008); and ϵ is the residual ϵ˜N(0,σ_{E}²).

gE represents the GEI effect and was equal to zero for the model without GEI (named GE). For the model that fitted GEI (named GxE), gE˜N(0,V_{gE}σ_{gE}²), V_{gE}=V_{g}⊙V_{E}, ⊙ is the Hadamard or cell-to-cell product. Both models were fitted in BGLR (Pérez and de Los Campos, 2014).

### Results

### Phenotypic Data

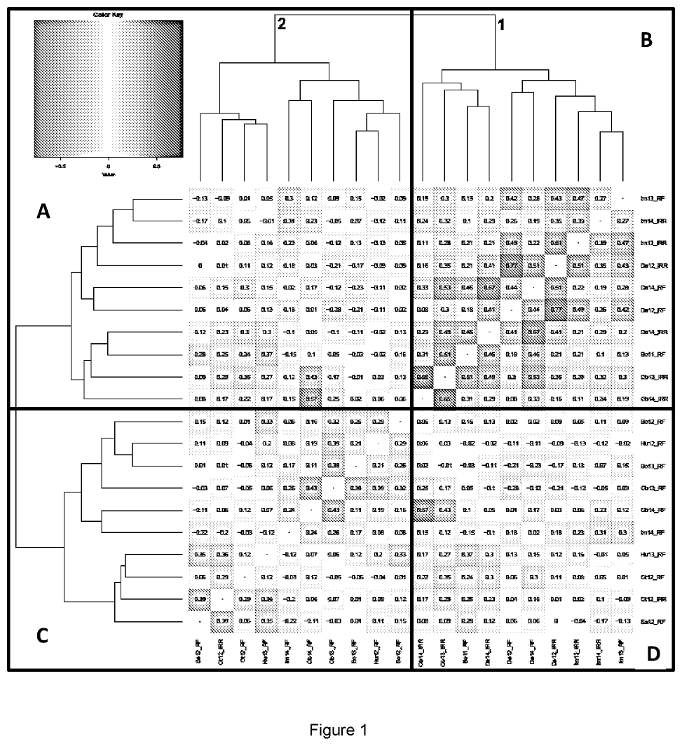

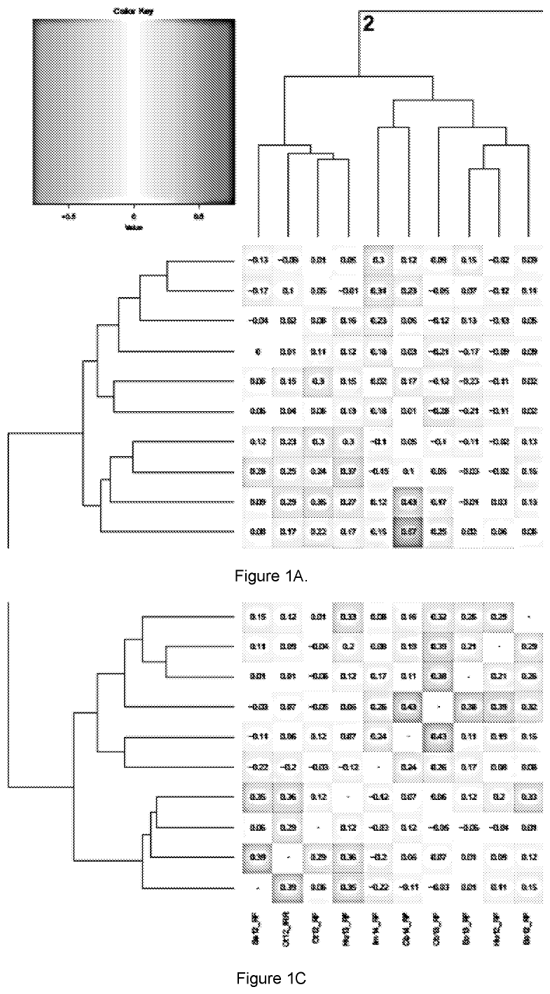

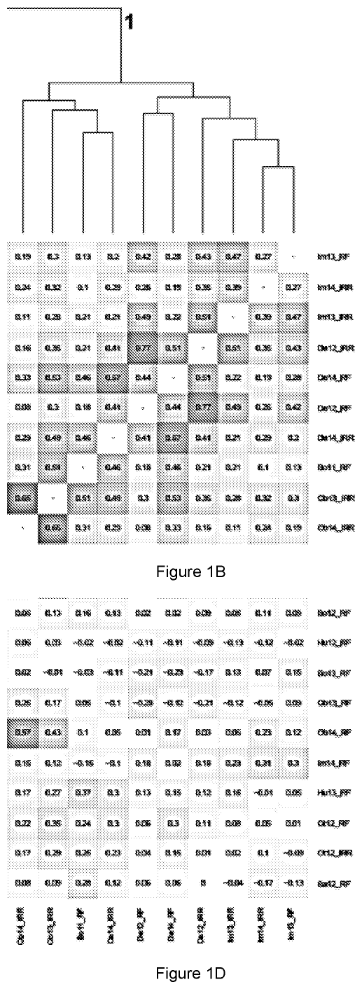

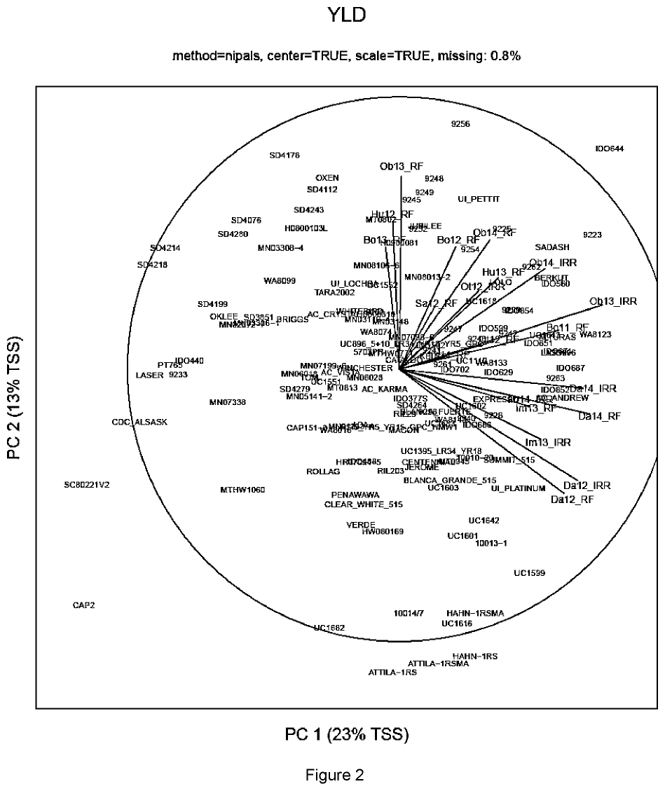

The twenty environments had a narrow sense heritability (h²) value ranging between 0.11 and 0.62 with an average of 0.31 and they were clustered in two major groups (Table 1; FIG. 1). The first cluster involved ten environments with average h² of 0.34 and pairwise phenotypic correlation coefficients ranging between 0.08 and 0.77 with an average of 0.34 (FIG. 1). Six of these environments had irrigation treatments, while the remaining had rain-fed treatments (Table 1). The second cluster also contained ten environments with lower average h² (0.29) and average phenotypic correlation coefficients (0.13) that ranged from −0.22 to 0.43. All of the environments of this cluster except one had rain-fed treatments (Table 1). The inter-cluster correlation coefficients had an average of 0.07 and ranged between −0.28 and 0.57. FIG. 2 showed the GGE biplot of the 144 reference individuals and the 20 environments. The first two principal components together explained 36% of the total variation.

TABLE 1
Calculating the phenotypic correlation coefficients for different environments, using the models GE, GxE and 3GS when using all
environments in the reference or when calculating unobserved environments. For unobserved environments: the phenotypic
correlation coefficients was calculated based on the pGEBVs of the environment with the highest correlation (BestCor; method 1);
average pGEBVs for same cluster (Mega; method 2) or full correlation (FullCor; method 3). Standard deviations for
different environments across methods ranged between 0.017 and 0.045, with average of 0.03.

| | | | Reference Environments | | | Unobserved Environments | | | | | |
|---|---|---|---|---|---|---|---|---|---|---|---|
| Trial | Cluster | h² | GE | GxE | 3GS | GE | GxE_BestCor | 3GS_BestCor | GxE_Mega | 3GS_Mega | 3GS_FullCor |
| Da12_RF | 1 | 0.558 | 0.206 | 0.241 | 0.237 | 0.198 | 0.298 | 0.291 | 0.169 | 0.144 | 0.171 |
| Da12_IRR | 1 | 0.495 | 0.343 | 0.348 | 0.314 | 0.336 | 0.249 | 0.241 | 0.33 | 0.299 | 0.303 |
| Da14_RF | 1 | 0.287 | 0.218 | 0.231 | 0.243 | 0.212 | 0.219 | 0.215 | 0.232 | 0.222 | 0.215 |
| Im13_RF | 1 | 0.138 | 0.06 | 0.059 | 0.044 | 0.06 | 0.028 | 0 | 0.038 | 0.006 | 0.026 |
| Im13_IRR | 1 | 0.307 | 0.139 | 0.281 | 0.233 | 0.112 | 0.183 | 0.135 | 0.119 | 0.07 | 0.113 |
| Im14_IRR | 1 | 0.288 | 0.034 | 0.123 | 0.089 | 0.018 | 0.074 | 0.044 | 0.012 | 0 | 0.037 |
| Bo11_RF | 1 | 0.332 | 0.306 | 0.36 | 0.383 | 0.294 | 0.22 | 0.21 | 0.278 | 0.282 | 0.303 |
| Da14_IRR | 1 | 0.405 | 0.255 | 0.23 | 0.215 | 0.251 | 0.236 | 0.238 | 0.246 | 0.245 | 0.271 |
| Ob13_IRR | 1 | 0.385 | 0.248 | 0.211 | 0.19 | 0.244 | 0.232 | 0.193 | 0.19 | 0.11 | 0.155 |
| Ob14_IRR | 1 | 0.187 | 0.155 | 0.183 | 0.172 | 0.147 | 0.12 | 0.085 | 0.076 | 0.026 | 0.154 |
| Bo12_RF | 2 | 0.318 | 0.089 | 0.271 | 0.358 | 0.08 | 0.27 | 0.325 | 0.188 | 0.267 | 0.26 |
| Bo13_RF | 2 | 0.145 | 0 | 0.083 | 0.14 | 0 | 0.11 | 0.17 | 0.044 | 0.169 | 0.271 |
| Hu12_RF | 2 | 0.191 | 0.146 | 0.147 | 0.138 | 0.145 | 0.186 | 0.209 | 0.186 | 0.222 | 0.067 |
| Im14_RF | 2 | 0.195 | 0 | 0 | 0.12 | 0 | 0 | 0.086 | 0.032 | 0.054 | 0 |
| Ob13_RF | 2 | 0.274 | 0.044 | 0.226 | 0.386 | 0.035 | 0.103 | 0.076 | 0.09 | 0.146 | 0.346 |
| Ob14_RF | 2 | 0.285 | 0.198 | 0.338 | 0.366 | 0.179 | 0.247 | 0.244 | 0.198 | 0.162 | 0.235 |
| Hu13_RF | 2 | 0.479 | 0.332 | 0.343 | 0.509 | 0.324 | 0.382 | 0.396 | 0.144 | 0.007 | 0.428 |
| Sa12_RF | 2 | 0.617 | 0 | 0.413 | 0.378 | 0 | 0.117 | 0.074 | 0.2 | 0.314 | 0.24 |
| Ot12_RF | 2 | 0.107 | 0.33 | 0.274 | 0.383 | 0.324 | 0.331 | 0.285 | 0.160 | 0.007 | 0.147 |
| Ot12_IRR | 2 | 0.264 | 0.182 | 0.15 | 0.152 | 0.173 | 0 | 0 | 0.030 | 0.085 | 0.171 |
| Mean | . | 0.338 | 0.196 | 0.227 | 0.212 | 0.187 | 0.186 | 0.165 | 0.169 | 0.14 | 0.175 |
| Cluster 1 | | | | | | | | | | | |
| Mean | . | 0.288 | 0.132 | 0.224 | 0.293 | 0.126 | 0.175 | 0.187 | 0.127 | 0.146 | 0.217 |
| Cluster 2 | | | | | | | | | | | |
| Mean | . | 0.313 | 0.164 | 0.226 | 0.252 | 0.157 | 0.18 | 0.176 | 0.148 | 0.143 | 0.196 |

### Genomic Selection for Reference Environments

The 3GS model was compared to the standard GBLUP model without (GE) and with (GxE) modelling GEI considering the 20 environments in the reference population. The results clearly demonstrated increased calculation accuracy when using 3GS compared to both GBLUP models. On average over all environments, applying 3GS increased the accuracy by 70% compared to the GE model and by 12% compared to the GxE model (0.252 for 3GS vs 0.164 for GE and 0.226 for GxE; Table 1). The calculation accuracy advantage occurred prominently in environments belonging to Cluster 2, where the accuracy of the 3GS model (r=0.293) was more than double that of the GE model (x=0.132) and was 31% higher than the GxE model (r=0.224). The average calculation accuracies of Cluster 1 environments were comparable between the 3GS, GE and GxE models: 0.217, 0.196 and 0.227, respectively (Table 1).

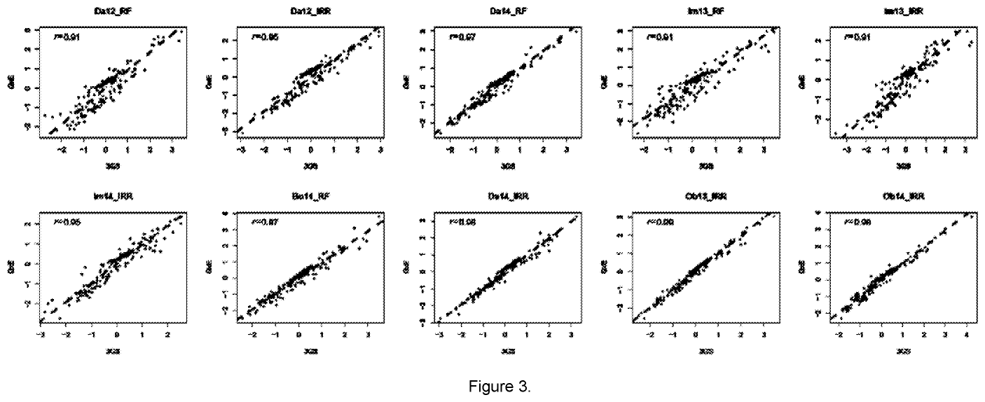

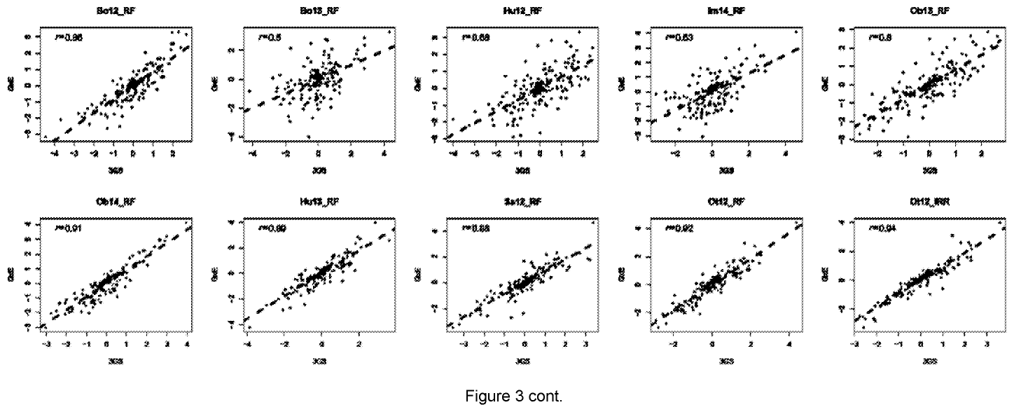

The pGEBV solutions produced by the 3GS model for environments within Cluster 1 were very comparable to the solutions produced by the GxE model. The average correlation coefficients between both models was 0.95, which ranged from 0.91 to 0.99 (FIG. 3). The calculation of the environments within Cluster 2 varied between both models with correlation coefficient values ranging from 0.5 to 0.94 and having an average of 0.8. The environments Bo13_RF, Im14_RF and Hu12_RF had correlation coefficients below the average: 0.5, 0.63 and 0.68, respectively (FIG. 3).

Comparisons of the correlations between pGEBVs produced by the 3GS and GxE models to the phenotypic correlations showed that the GxE model tended to overestimate the correlation among environments, while 3GS produced more realistic estimates (Tables 2A-C). In Tables 2A-C, positive values indicate positive pairwise correlations, and negative values indicate negative pairwise correlations. The depth of shading is indicative of the strength of correlation, with deeper shades representing stronger pairwise correlations. A positive correlation indicates that genotypes in both environments have the same phenotype. For example, genotypes that have high phenotypes in one environment would be expected to have high phenotypes in another environment. The higher the correlation value, the stronger the relationship between the environments. On the other hand, negative correlations indicate the reverse. For example, genotypes that have high phenotypes in one environment are expected to have a low phenotype in another environment. Almost all pairwise correlations for the pGEBVs of the GxE model were higher than those of the phenotypic data, with an average increase of 0.35. Environments in Cluster 1 showed a higher average increase (0.43) compared to environments in Cluster 2 (0.32). The pGEBVs of the 3GS model showed higher correlations only for environments within Cluster 1 (average 0.26 increase), while differences for Cluster 2 and inter-cluster correlations ranged from −0.41 to 0.65, with an average of zero. The average absolute differences between the correlations of the pGEBVs of the 3GS model and the phenotypic correlations was equal to 0.21, which was smaller than that of the GxE model (inferred from Table 2C to be equal to 0.35).

TABLE 2A
Pairwise correlations between different environments for the phenotypic data model.

| pheno | Da12_RF | Da12_IRR | Da14_RF | Im13_RF | Im13_IRR | Im14_IRR | Bo11_RF | Da14_IRR | Ob13_IRR | Ob14_IRR | Bo12_RF |
|---|---|---|---|---|---|---|---|---|---|---|---|
| Da12_RF | 1 | 0.7722 | 0.435474 | 0.419548 | 0.489582 | 0.255519 | 0.177614 | 0.406754 | 0.299108 | 0.078724 | 0.020277 |
| Da12_IRR | 0.7722 | 1 | 0.507958 | 0.427486 | 0.507823 | 0.349043 | 0.21236 | 0.408507 | 0.34897 | 0.157398 | 0.086388 |
| Da14_RF | 0.435474 | 0.507958 | 1 | 0.281013 | 0.218864 | 0.19382 | 0.456951 | 0.666199 | 0.528303 | 0.330887 | 0.024845 |
| Im13_RF | 0.419548 | 0.427486 | 0.281013 | 1 | 0.466665 | 0.269571 | 0.129058 | 0.204805 | 0.301444 | 0.187474 | 0.08765 |
| Im13_IRR | 0.489582 | 0.507823 | 0.218864 | 0.466665 | 1 | 0.39315 | 0.210812 | 0.205197 | 0.275273 | 0.109222 | 0.045742 |
| Im14_IRR | 0.255519 | 0.349043 | 0.19382 | 0.269571 | 0.39315 | 1 | 0.098364 | 0.285084 | 0.317082 | 0.241963 | 0.113064 |
| Bo11_RF | 0.177614 | 0.21236 | 0.456951 | 0.129058 | 0.210812 | 0.098364 | 1 | 0.460361 | 0.509562 | 0.311421 | 0.157251 |
| Da14_IRR | 0.406754 | 0.408607 | 0.666199 | 0.204805 | 0.205197 | 0.285084 | 0.460361 | 1 | 0.490929 | 0.292227 | 0.131357 |
| Ob13_IRR | 0.299108 | 0.34897 | 0.528303 | 0.301444 | 0.275273 | 0.317082 | 0.509562 | 0.490929 | 1 | 0.649923 | 0.130953 |
| Ob14_IRR | 0.078724 | 0.157398 | 0.330887 | 0.187474 | 0.109222 | 0.241963 | 0.311421 | 0.292227 | 0.649923 | 1 | 0.056092 |
| Bo12_RF | 0.020277 | 0.086388 | 0.024845 | 0.08765 | 0.045742 | 0.113064 | 0.157251 | 0.131357 | 0.130953 | 0.056092 | 1 |
| Bo13_RF | [symbol] | [symbol] | [symbol] | 0.15045 | 0.128899 | 0.072022 | −0.02712 | −0.11126 | −0.01467 | 0.018814 | 0.261252 |
| Hu12_RF | −0.1066 | −0.09378 | −0.11037 | −0.02253 | −0.1323 | −0.12174 | −0.01643 | −0.02012 | 0.026474 | 0.06378 | 0.289171 |
| Im14_RF | 0.176577 | 0.183243 | 0.024374 | 0.299741 | 0.231109 | 0.306741 | −0.14879 | −0.09644 | 0.120185 | 0.151142 | 0.082074 |
| Ob13_RF | [symbol] | [symbol] | −0.11583 | 0.090083 | −0.11768 | −0.05476 | 0.046763 | −0.10322 | 0.16897 | 0.246264 | 0.315411 |
| Ob14_RF | 0.012207 | 0.028289 | 0.173123 | 0.123797 | 0.063512 | 0.225686 | 0.098924 | 0.045872 | 0.427474 | 0.57214 | 0.163704 |
| Hu13_RF | 0.131628 | 0.118634 | 0.150498 | 0.050136 | 0.160539 | −0.00818 | 0.367254 | 0.304041 | 0.267253 | 0.174101 | 0.331336 |
| Sa12_RF | 0.059822 | −0.00463 | 0.06436 | −0.13468 | −0.03894 | −0.16754 | 0.283826 | 0.120347 | 0.094877 | 0.081715 | 0.149602 |
| Ot12_RF | 0.064935 | 0.108366 | 0.299692 | 0.009758 | 0.081477 | 0.051294 | 0.241012 | 0.298983 | 0.346539 | 0.222068 | 0.013126 |
| Ot12_IRR | 0.036427 | 0.013366 | 0.151668 | −0.09495 | 0.023686 | 0.099303 | 0.24888 | 0.231369 | 0.285906 | 0.173432 | 0.116036 |
| | pheno | Bo12_RF | Bo13_RF | Hu12_RF | Im14_RF | Ob13_RF | Ob14_RF | Hu13_RF | Sa12_RF | Ot12_RF | Ot12_IRR |
| | Da12_RF | 0.020277 | −0.221178 | −0.1066 | 0.176577 | [symbol] | 0.012207 | 0.131628 | 0.059822 | 0.064935 | 0.036427 |
| | Da12_IRR | 0.086388 | [symbol] | −0.09378 | 0.183243 | [symbol] | 0.028289 | 0.118634 | −0.00463 | 0.108366 | 0.013366 |
| | Da14_RF | 0.024845 | [symbol] | −0.11037 | 0.024374 | −0.11583 | 0.173123 | 0.150498 | 0.06436 | 0.299692 | 0.151668 |
| | Im13_RF | 0.08765 | 0.15045 | −0.02253 | 0.299741 | 0.090083 | 0.123797 | 0.050136 | −0.13438 | 0.009758 | −0.09495 |
| | Im13_IRR | 0.045742 | 0.128899 | −0.1323 | 0.231109 | −0.11768 | 0.063512 | 0.160539 | −0.03894 | 0.081477 | 0.023686 |
| | Im14_IRR | 0.113064 | 0.072022 | −0.12174 | 0.306741 | −0.05476 | 0.225686 | −0.00818 | [symbol] | 0.051294 | 0.099303 |
| | Bo11_RF | 0.157251 | −0.02712 | −0.01643 | −0.14879 | 0.046763 | 0.098924 | 0.367254 | 0.283826 | 0.241012 | 0.24888 |
| | Da14_IRR | 0.131357 | 0.11126 | −0.02012 | 0.09644 | 0.10322 | 0.045872 | 0.304041 | 0.120347 | 0.98983 | 0.231369 |
| | Ob13_IRR | 0.130953 | −0.01467 | 0.026474 | 0.120185 | 0.16897 | 0.427474 | 0.267253 | 0.094877 | 0.346539 | 0.285906 |
| | Ob14_IRR | 0.056092 | 0.018814 | 0.06378 | 0.151142 | 0.246254 | 0.57214 | 0.174101 | 0.081715 | 0.222068 | 0.173432 |
| | Bo12_RF | 1 | 0.261252 | 0.289171 | 0.082074 | 0.315411 | 0.163704 | 0.331336 | 0.149602 | 0.013126 | 0.116036 |
| | Bo13_RF | 0.261252 | 1 | 0.212619 | 0.172783 | 0.375566 | 0.105884 | 0.122677 | 0.012234 | −0.0611 | 0.005055 |
| | Hu12_RF | 0.289171 | 0.212619 | 1 | 0.079738 | 0.391306 | 0.186044 | 0.204393 | 0.113785 | −0.04209 | 0.086799 |
| | Im14_RF | 0.082074 | 0.172783 | 0.079738 | 1 | 0.260091 | 0.239109 | 0.12429 | −0.22101 | −0.03107 | −0.20213 |
| | Ob13_RF | 0.315411 | 0.375566 | 0.391306 | 0.260091 | 1 | 0.432796 | 0.056807 | −0.0303 | −0.04596 | 0.069368 |
| | Ob14_RF | 0.163704 | 0.105884 | 0.186044 | 0.239109 | 0.432796 | 1 | 0.07182 | −0.10855 | 0.117075 | 0.060197 |
| | Hu13_RF | 0.331336 | 0.122677 | 0.204393 | −0.12429 | 0.056807 | 0.07182 | 1 | 0.351317 | 0.124252 | 0.355317 |
| | Sa12_RF | 0.149602 | 0.012234 | 0.113785 | −0.22101 | −0.0303 | −0.10855 | 0.351317 | 1 | 0.063234 | 0.39302 |
| | Ot12_RF | 0.013126 | −0.0611 | −0.04209 | −0.03107 | −0.04596 | 0.117075 | 0.124252 | 0.063234 | 1 | 0.288036 |
| | Ot12_IRR | 0.116036 | 0.005055 | 0.086799 | −0.20213 | 0.069368 | 0.060197 | 0.355317 | 0.39302 | 0.288036 | 1 |

[symbol]

indicates data missing or illegible when filed

TABLE 2B
Pairwise correlations between different environments for the pGEBVs produced using 3GS model.

| pheno | Da12_RF | Da12_IRR | Da14_RF | Im13_RF | Im13_IRR | Im14_IRR | Bo11_RF | Da14_IRR | Ob13_IRR | Ob14_IRR |
|---|---|---|---|---|---|---|---|---|---|---|
| Da12_RF | 1 | 0.694318 | 0.448409 | [symbol] | [symbol] | 0.375288 | 0.266264 | 0.440518 | 0.374513 | 0.401964 |
| Da12_IRR | [symbol] | 1 | 0.700078 | 0.0600974 | 0.602427 | 0.740584 | 0.495092 | 0.5602055 | 0.577237 | [symbol] |
| Da14_RF | 0.448409 | 0.700078 | 1 | 0.666969 | 0.545314 | 0.605912 | 0.694546 | 0.66174 | 0.85231 | 0.766625 |
| Im13_RF | 0.556023 | 0.500974 | 0.666969 | 1 | 0.555323 | 0.364856 | 0.560203 | 0.702923 | 0.524063 | 0.564416 |
| Im13_IRR | 0.485047 | 0.602427 | 0.545314 | 0.555323 | 1 | 0.559657 | 0.553129 | 0.536266 | [symbol] | 0.487961 |
| Im14_IRR | 0.375228 | 0.740584 | 0.605932 | 0.364856 | 0.559657 | 1 | 0.514754 | 0.594182 | 0.689725 | 0.583001 |
| Bo11_RF | 0.266264 | 0.495092 | [symbol] | 0.560203 | 0.553129 | 0.514754 | 1 | 0.715402 | 0.725769 | 0.512609 |
| Da14_IRR | 0.440518 | 0.662055 | 0.86174 | 0.702923 | 0.536266 | 0.594182 | [symbol] | 1 | 0.790625 | [symbol] |
| Ob13_IRR | 0.374513 | 0.677237 | 0.85231 | 0.624063 | 0.514565 | 0.689725 | 0.725769 | 0.790625 | 1 | 0.833654 |
| Ob14_IRR | 0.401964 | 0.613635 | 0.766626 | 0.564416 | 0.487961 | 0.583001 | 0.513309 | 0.695558 | 0.833654 | 1 |
| Bo12_RF | −0.31229 | −0.13154 | −0.18971 | −0.12399 | [symbol] | −0.10878 | −0.10359 | −0.09046 | −0.20626 | −0.16164 |
| Bo13_RF | −0.29966 | −0.38311 | −0.33167 | −0.19189 | 0.11484 | −0.22925 | −0.04077 | −0.14836 | −0.19105 | −0.1828 |
| Hu12_RF | −0.24005 | −0.3074 | −0.33166 | −0.28415 | [symbol] | [symbol] | −0.3217 | −0.30805 | [symbol] | −0.19129 |
| Im14_RF | 0.248329 | 0.015481 | 0.012725 | 0.140366 | 0.039926 | −0.05544 | −0.19233 | 0.009605 | 0.07951 | 0.187314 |
| Ob13_RF | −0.32714 | [symbol] | −0.18577 | −0.10139 | [symbol] | [symbol] | −0.11871 | −0.1287 | −0.17127 | −0.03421 |
| Ob14_RF | 0.108254 | 0.326872 | 0.455713 | 0.264029 | 0.075427 | 0.218928 | 0.144946 | 0.365796 | 0.487451 | 0.67784 |
| Hu13_RF | 0.182242 | 0.260959 | 0.245692 | 0.178213 | 0.308295 | 0.201347 | 0.512825 | 0.384701 | 0.256612 | 0.192812 |
| Sa12_RF | 0.221113 | 0.181374 | 0.090787 | 0.022691 | 0.072238 | 0.142188 | −0.06214 | −0.01968 | 0.221012 | 0.354989 |
| Ot12_RF | 0.287467 | 0.38576 | 0.555974 | 0.44443 | 0.453793 | 0.474228 | [symbol] | 0.548054 | 0.607648 | 0.466744 |
| Ot12_IRR | 0.311171 | 0.616091 | 0.434782 | 0.291144 | 0.518973 | 0.745544 | 0.525865 | 0.42045 | 0.632623 | 0.584033 |
| pheno | Bo12_RF | Bo13_RF | Hu12_RF | Im14_RF | Ob13_RF | Ob14_RF | Hu13_RF | Sa12_RF | Ot12_RF | Ot12_IRR |
| Da12_RF | −0.31229 | 0.29966 | −0.24005 | 0.248329 | −0.32714 | 0.108254 | 0.182242 | 0.221113 | 0.287457 | 0.311171 |
| Da12_IRR | −0.13154 | [symbol] | −0.3074 | 0.015481 | [symbol] | 0.326872 | 0.260959 | 0.181374 | 0.38576 | 0.616001 |
| Da14_RF | −0.18971 | −0.33167 | −0.33166 | 0.012725 | −0.18577 | 0.455713 | 0.245692 | 0.090787 | 0.555974 | 0.434782 |
| Im13_RF | −0.12399 | −0.19189 | −0.28415 | 0.140366 | −0.10139 | 0.264029 | 0.178213 | 0.022691 | 0.44443 | 0.291144 |
| Im13_IRR | −0.33406 | −0.11464 | 0.35886 | 0.039926 | [symbol] | 0.075427 | 0.308295 | 0.073238 | 0.453793 | 0.518973 |
| Im14_IRR | −0.10878 | −0.22935 | −0.34897 | −0.05544 | [symbol] | 0.218928 | 0.201347 | 0.142188 | 0.474228 | 0.745544 |
| Bo11_RF | −0.10359 | −0.04077 | −0.3217 | −0.19233 | −0.11871 | 0.144946 | 0.512825 | −0.06214 | 0.595217 | 0.525865 |
| Da14_IRR | −0.09046 | −0.14836 | −0.30805 | 0.009605 | −0.1287 | 0.365796 | 0.384701 | −0.01968 | 0.548054 | 0.42045 |
| Ob13_IRR | −0.20626 | −0.19105 | [symbol] | 0.07951 | −0.17127 | 0.487451 | 0.56612 | 0.221012 | 0.607648 | 0.632623 |
| Ob14_IRR | −0.16164 | −0.1828 | −0.19129 | 0.187314 | −0.03421 | 0.67784 | 0.192812 | 0.354989 | 0.466744 | [symbol] |
| Bo12_RF | 1 | 0.282144 | 0.564537 | −0.11385 | 0.444735 | 0.237783 | 0.279111 | −0.00758 | −0.16678 | −0.22663 |
| Bo13_RF | 0.282144 | 1 | 0.429844 | 0.076282 | 0.51454 | −0.0033 | 0.240991 | 0.129837 | −0.07979 | −0.0411 |
| Hu12_RF | 0.564537 | 0.429844 | 1 | −0.10928 | 0.553833 | 0.212858 | 0.24803 | 0.225731 | [symbol] | −0.25085 |
| Im14_RF | −0.11385 | 0.076282 | −0.10928 | 1 | 0.121642 | 0.095573 | −0.17721 | 0.339153 | 0.041136 | −0.02956 |
| Ob13_RF | 0.444735 | 0.51454 | 0.553833 | 0.121642 | 1 | 0.313862 | 0.186137 | 0.063227 | −0.10801 | −0.22052 |
| Ob14_RF | 0.237783 | −0.0033 | 0.212858 | 0.095573 | 0.313862 | 1 | 0.199541 | 0.37991 | 0.060686 | 0.255223 |
| Hu13_RF | 0.279111 | −0.24099 | 0.24803 | −0.17721 | 0.186137 | 0.199541 | 1 | −0.04405 | 0.231078 | 0.263632 |
| Sa12_RF | −0.00758 | 0.129837 | 0.225731 | 0.339153 | 0.063227 | 0.37991 | −0.04405 | 1 | −0.13283 | 0.339281 |
| Ot12_RF | −0.16678 | −0.07979 | [symbol] | 0.045136 | −0.10801 | 0.060686 | 0.231078 | −0.13283 | 1 | 0.359928 |
| Ot12_IRR | 0.22663 | −0.0411 | 0.25085 | −0.02956 | −0.22052 | 0.255223 | 0.263632 | 0.339281 | 0.359928 | 1 |

[symbol]

indicates data missing or illegible when filed

TABLE 2C
Pairwise correlations between different environments for the GxE model.

| pheno | Da12_RF | Da12_IRR | Da14_RF | Im13_RF | Im13_IRR | Im14_IRR | Bo11_RF | Da14_IRR | Ob13_IRR | Ob14_IRR |
|---|---|---|---|---|---|---|---|---|---|---|
| Da12_RF | 1 | [symbol] | 0.741257 | [symbol] | 0.77444 | 0.716121 | [symbol] | 0.70179 | 0.713178 | [symbol] |
| Da12_IRR | [symbol] | 1 | 0.817612 | [symbol] | [symbol] | 0.824757 | 0.655136 | 0.775171 | 0.846297 | 0.793307 |
| Da14_RF | 0.741257 | 0.817612 | 1 | 0.795294 | 0.743834 | [symbol] | 0.613744 | [symbol] | 0.908711 | 0.844233 |
| Im13_RF | [symbol] | 0.640314 | 0.795294 | 1 | [symbol] | 0.733858 | 0.649157 | 0.761076 | 0.525918 | 0.76502 |
| Im13_IRR | 0.77444 | [symbol] | 0.743894 | 0.826236 | 1 | [symbol] | 0.667302 | 0.691557 | 0.823963 | 0.735851 |
| Im14_IRR | 0.716121 | 0.874757 | [symbol] | [symbol] | [symbol] | 1 | 0.587243 | [symbol] | [symbol] | [symbol] |
| Bo11_RF | 0.369593 | 0.655136 | 0.813744 | 0.649157 | 0.667302 | 0.567243 | 1 | 0.819985 | 0.773217 | 0.638943 |
| Da14_IRR | 0.70179 | 0.776173 | [symbol] | 0.761076 | 0.691667 | 0.677648 | [symbol] | 1 | 0.850097 | 0.778352 |
| Ob13_IRR | 0.713178 | 0.846297 | 0.903711 | [symbol] | [symbol] | 0.83523 | 0.773217 | 0.850097 | 1 | [symbol] |
| Ob14_IRR | 0.689369 | 0.793307 | 0.844233 | 0.76502 | [symbol] | 0.765634 | 0.638943 | [symbol] | 0.005998 | 1 |
| Bo12_RF | 0.102002 | 0.204272 | 0.155083 | 0.219023 | 0.108314 | 0.137807 | 0.224046 | 0.217578 | 0.144699 | 0.189262 |
| Bo13_RF | 0.421828 | 0.454813 | 0.381731 | 0.479121 | 0.492119 | 0.440683 | 0.405919 | 0.411806 | 0.431388 | 0.461655 |
| Hu12_RF | 0.215214 | 0.228774 | 0.225308 | 0.208932 | 0.180498 | 0.140379 | 0.22451 | 0.235243 | 0.205221 | 0.321577 |
| Im14_RF | 0.662267 | 0.69329 | 0.57023 | 0.735128 | 0.686852 | 0.651988 | 0.35492 | 0.496505 | 0.673356 | 0.694452 |
| Ob13_RF | 0.073022 | 0.121858 | 0.211499 | 0.274695 | 0.126528 | 0.078148 | 0.19169 | 0.192677 | 0.256165 | 0.336427 |
| Ob14_RF | 0.517855 | 0.637739 | 0.657616 | 0.62883 | [symbol] | 0.580584 | 0.430859 | 0.585182 | 0.722826 | [symbol] |
| Hu13_RF | 0.411501 | 0.404136 | 0.458144 | 0.325024 | 0.379533 | 0.275703 | 0.619359 | 0.528455 | 0.403903 | 0.395016 |
| Sa12_RF | 0.166882 | 0.086744 | 0.087205 | [symbol] | [symbol] | −0.00596 | 0.089266 | 0.097809 | 0.082402 | 0.236357 |
| Ot12_RF | 0.625238 | 0.654656 | 0.777241 | 0.665359 | [symbol] | 0.666688 | [symbol] | 0.75016 | 0.764086 | 0.680112 |
| Ot12_IRR | 0.535226 | 0.631392 | 0.495468 | 0.417858 | 0.573523 | 0.678925 | [symbol] | 0.499439 | 0.596873 | 0.603576 |
| pheno | Bo12_RF | Bo13_RF | Hu12_RF | Im14_RF | Ob13_RF | Ob14_RF | Hu13_RF | Sa12_RF | Ot12_RF | Ot12_IRR |
| Da12_RF | 0.102002 | 0.421828 | 0.215214 | 0.662267 | 0.073022 | [symbol] | 0.411501 | 0.166882 | [symbol] | 0.535226 |
| Da12_IRR | 0.204272 | 0.454813 | 0.228774 | 0.69329 | 0.121858 | [symbol] | 0.404136 | 0.086744 | [symbol] | 0.631397 |
| Da14_RF | 0.155083 | 0.381731 | 0.225308 | 0.57023 | 0.211499 | [symbol] | 0.458144 | 0.087205 | 0.777241 | 0.495468 |
| Im13_RF | 0.219023 | 0.479121 | 0.208932 | 0.735128 | 0.274695 | [symbol] | 0.325024 | −0.02299 | 0.665359 | 0.417868 |
| Im13_IRR | 0.108347 | 0.492119 | 0.180498 | 0.685862 | 0.126528 | 0.559911 | 0.379533 | −0.01678 | 0.685841 | 0.573523 |
| Im14_IRR | 0.137807 | 0.440683 | 0.140379 | 0.651988 | 0.078148 | [symbol] | 0.275703 | −0.00596 | 0.666688 | 0.678925 |
| Bo11_RF | 0.224046 | 0.405919 | 224510.0 | 0.35492 | 0.19169 | 0.430659 | [symbol] | 0.089265 | 0.739685 | 0.550901 |
| Da14_IRR | 0.217578 | 0.411806 | 0.235243 | 0.496505 | 0.192577 | 0.585182 | 0.528455 | 0.097809 | 0.75016 | 0.499439 |
| Ob13_IRR | 0.144699 | 0.431388 | 0.205221 | 0.673356 | 0.256165 | 0.722826 | 0.403903 | 0.082402 | 0.764086 | 0.596873 |
| Ob14_IRR | 0.189262 | 0.461665 | 0.321577 | 0.694462 | 0.336427 | [symbol] | 0.395016 | 0.236357 | 0.680112 | 0.603676 |
| Bo12_RF | 1 | 0.655018 | 0.753311 | 0.307654 | 0.662131 | 0.45013 | [symbol] | 0.315837 | 0.195477 | 0.127709 |
| Bo13_RF | 0.655018 | 1 | 0.76467 | 0.628927 | 0.712235 | 0.577013 | [symbol] | 0.408635 | 0.444098 | 0.470581 |
| Hu12_RF | 0.753311 | 0.76467 | 1 | 0.345597 | 0.720234 | 0.547231 | 0.681215 | 0.519805 | 0.170561 | 0.271794 |
| Im14_RF | 0.307654 | 0.628927 | 0.345597 | 1 | 0.450122 | 0.669824 | 0.260691 | 0.212009 | 0.513459 | 0.402154 |
| Ob13_RF | 0.662131 | 0.712235 | 0.720234 | 0.450122 | 1 | [symbol] | 0.473859 | 0.67018 | 0.243141 | 0.139235 |
| Ob14_RF | 0.45013 | 0.577013 | 0.547231 | 0.669824 | [symbol] | 1 | 0.438392 | 0.291035 | 0.472439 | 0.430223 |
| Hu13_RF | [symbol] | 0.658652 | 0.681215 | 0.260691 | 0.473859 | 0.438392 | 1 | 0.413039 | 0.459747 | [symbol] |
| Sa12_RF | 0.315837 | 0.408635 | 0.519805 | 0.212009 | 0.267018 | 0.291035 | 0.413039 | 1 | [symbol] | 0.407359 |
| Ot12_RF | 0.195477 | 0.444098 | 0.170561 | 0.513459 | 0.243141 | 0.472439 | 0.459747 | −0.01872 | 1 | 0.476495 |
| Ot12_IRR | 0.127709 | 0.470581 | 0.271794 | 0.402154 | 0.139235 | 0.430223 | 0.505328 | 0.407359 | 0.476495 | 1 |

[symbol]

indicates data missing or illegible when filed

### Accuracy for Calculating Unobserved Environments

Omitting one environment from the reference population at a time showed that 3GS can calculate new environments with good accuracy. As the initial models for 3GS and GxE only produce pGEBVs for the reference environments, three novel methods were assessed to calculate new environments. The first and third methods further increased overall calculation accuracy for both the GxE and 3GS models, compared to the GE. This improvement resulted mainly from higher calculation accuracy for environments in Cluster 2 (Table 1). The first method is the simplest to implement because it directly calculates the accuracy of the unobserved environment from the pGEBVs of the reference environment that has the highest correlation with it. This method performed comparable in both the GxE and 3GS models, with an average calculation accuracy of 0.180 and 0.176, respectively (Table 1). The second method calculates the mean of pGEBVs within each cluster of environments ‘or mega environment’ for each individual. The first method calculated new environments more accurately than this second method (Table 1). The third method assumes that the pGEBVs for the unobserved environment can be calculated from its correlation with the reference environments, as well as the GEBVs of the GGE principal components for the reference environments. For this reason, it is specific to 3GS model. This method showed the highest average accuracy for calculating new environments (average r=0.196) compared to the other two methods, regardless of whether they were applied on the GxE or 3GS model (Table 1).

### Computational Requirements

The 3GS model was computationally very efficient in terms of memory and time requirements. Calculating each PC required less than 30 seconds and is a process that can be easily parallelized. Hence, if the number of threads was equal to the number of environments, the entire analysis would require the same amount of time needed to analyze a single PC. The analysis also required a maximum of 2.6 GB of RAM per thread which is slightly larger than the size of the genotypic data. In contrast, the GE model required slightly less than 3.5 minutes and 2.6 GB of RAM to run, while the GxE model required around 40 minutes and a maximum memory of 4.5 GB.

### Discussion

Several models have previously been proposed to fit GEI with genomic prediction. Burgueño et al. (2012) were the first to fit genetic correlation with GBLUP to account for GEI in crops, while Jarquin et al. (2014) were first to extend the GBLUP model to fit environmental covariates and their interactions with genetic variants. The linear GBLUP model proposed by López-Cruz et al. (2015) decomposes variant effects into main or stability effects and environment-specific deviations, while Cuevas et al. (2016) implemented the same model in a nonlinear Gaussian Kernel (GK) framework. GK models are less practical because they capture epistatic effects that get disrupted over generations (He et al. 2017; Jiang et al. 2018). The latter two models assume the environments are positively correlated because the correlation is inferred as a proportion of the total variation, which makes them inefficient for uncorrelated and negatively correlated environments. Cuevas et al. (2017) modeled genetic effects as the Kronecker produce of the correlations between environments and the genomic relatedness matrix. This approach showed comparable accuracies to the model proposed by Burgueño et al. (2012) when implemented in the GBLUP method. They also extended the model with a parameter representing the random effect among environments, which improved the calculation accuracy. However, this parameter cannot be applied to new genotypes that are not included in the training population, which limits its application in breeding programs. Compared to these previously described models, 3GS offers several advantages in terms of the complexity of the correlation structure among environments, ability to calculate new genotypes in unobserved environments and computational resource requirements.

### Benefits of the 3GS Model

### Calculation Accuracy of 3GS for Unobserved Genotypes in Reference Environments

The 3GS model gave higher calculation accuracy compared to the GxE model for environments that are less related to other environments in the reference. 3GS is therefore more robust in calculating complex interactions of quantitative trait loci with environments (Hayes et al. 2016). This was further confirmed by the ability of 3GS model to produce pGEBVs with comparable pairwise correlation values to those calculated using the original phenotypic data. In contrast, the GxE model consistently overestimated the relatedness among environments and flipped negatively correlated environments into positively correlated ones. Another advantage of the 3GS model is the calculation of the principal components of the GGE analysis, which allows all phenotyped and unphenotyped individuals in a GGE biplot to be compared for better selection decisions.

### Calculating Unobserved Genotypes in Unobserved Environments

One of the main difficulties for GS models that fit GEI is the ability to calculate unobserved genotypes in unobserved environments. Most previously published models calculate their accuracies by calculating the performance of new genotypes in the reference environments or by calculating incomplete field trials only (Burgueño et al. 2012; Jarquin et al. 2014; López-Cruz et al. 2015; Cuevas et al. 2016, 2017). Jarquin et al. (2017) described different models to exploit GEI that allowed calculation of unobserved environments, but the accuracies of their models were very low when calculating new genotypes in new environments. In contrast, 3GS is very promising for calculating the performance of populations in new environments or future climates. The third method as detailed above for calculating unobserved environments, and which is specific to the 3GS model, showed an enhanced performance compared to the other two methods applied to both models. The concept behind this method assumes that the variance of the extra PC representing the special variance component of the new environment is equal to zero; in other words, it assumes that the new environment does not add any new variation to the dataset so it will be completely dependent on the reference environments given its correlation with them.

The first method for calculating new environments as detailed above was more biased than the third method because it infers its calculation from only one environment (the new environment that has the highest correlation with the target new environment) which might not be the true calculator of the unobserved environment. This bias was noticed in the data as the method calculated many environments with zero accuracy, despite being well calculated with the other methods (Table 1). For this reason, implementing the third method to calculate new environments in breeding programs is recommended. The second method did not perform as well on the current dataset. However, in very large multi-environmental trials where each mega-environment is well represented in the reference dataset and distinguished from other mega-environments, this method could have better accuracy.

### Computational Efficiency of the 3GS Model

In general, the complexity for analyzing multi-environmental trials increases exponentially when moving from a univariate model (single environment) to a multivariate (multi environment) model. The R package BGGE (Granato et al. 2018) exploits the sparsity of covariance matrices to reduce the computational demand and was shown to be up to five times faster than the classical solver implemented in the R package BGLR (Pérez and de Los Campos, 2014). The multi-trait deep learning (MTDL) model proposed by Montesinos-López et al. (2018) can be parallelized to reduce computational time, while the variational Bayes model (BVM) proposed by Montesinos-López et al. (2017) was around 10 times faster than conventional Bayesian approaches. Nevertheless, each of these models is still computationally demanding due to existence of higher dimensionality compared to the univariate models. In contrast, calculating each PC in 3GS is equivalent to running a univariate GS model, meaning that the complexity of the analysis increases linearly with an increasing number of environments. This makes the 3GS model highly efficient from a computational standpoint in terms of both memory and time requirements. Moreover, due to the independency of the GGE PCs, 3GS model can be easily parallelized and with enough CPU cores, the total computational time can be reduced to the time required to analyze a single environment. The ability to calculate SNP effects for the PCs of GGE analysis also make it easier to calculate new genotypes using these effects, instead of repeating the entire analysis as in GBLUP (Lourenco et al. 2015).

The 3GS model runs optimally for a ‘semi-balanced’ dataset across environments. However, while the reference population is expected to have phenotypic records in all environments, PC imputation algorithms such as the nonlinear iterative partial least squares (NIPALS) can be used to infer some missing phenotypic data in 3GS with minimal effect on calculation accuracy. Preda et al. (2010) reported that less than 5% of missing data had no significant effect, while up to 15% of missing data had an acceptable effect on the accuracy of calculating different PCs. Sattari et al. (2017) showed that imputing 10% missing precipitation data with NIPALS method had an accuracy of 0.94.

### Conclusion

A novel computational model called 3GS has been developed that combines genomic selection with genotype plus genotype×environment interaction (GGE) analysis. The new model improved calculation accuracy above previously reported models that exploit GEI. It also has more elasticity to model complex relationships among environments without inflating the correlation coefficients and does not appear to be impacted by negative correlations among environments. Unlike previous models that consider GEI, 3GS is sufficiently flexible to calculate new genotypes in unobserved environments with good accuracy. Moreover, 3GS has a computational advantage over existing models, especially for massive datasets, because its complexity increases linearly with an increasing the number of environments. For this reason, 3GS can be optimally applied in current modern breeding programs where massive populations get screened in multiple environments or seasons with high-throughput phenotyping techniques.

Finally, it is to be understood that various alterations, modifications and/or additions may be made without departing from the spirit of the present invention as outlined herein.

### REFERENCES

Braun, H. J., Rajaram, S., & Ginkel, M. (1997). CIMMYT's approach to breeding for wide adaptation. Euphytica 92, 175-183

Burgueño, J., de los Campos, G., Weigel, K., & Crossa, J. (2012). Genomic prediction of breeding values when modeling genotype×environment interaction using pedigree and dense molecular markers. Crop Science, 52(2), 707-719
Butler, D., Cullis, B. R., Gilmour, A. R., & Gogel B. J. (2009). ASREML-R, Reference Manual Version 3. Queensland Department of Primary Industries and Fisheries, Brisbane
Crossa, J., Pérez-Rodriguez, P., Cuevas, J., Montesinos-Lopez, O., Jarquin, D., de los Campos, G., et al. (2017). Genomic selection in plant breeding: methods, models, and perspectives. Trends in plant science, 22(11), 961-975

Cuevas, J., Crossa, J., Soberanis, V., Pérez-Elizalde, S., Perez-Rodriguez, P., Campos, G. D. L., et al. (2016). Genomic prediction of genotype×environment interaction kernel regression models. The plant genome, 9(3), 1-20
Cuevas, J., Crossa, J., Montesinos-López, O. A., Burgueño, J., Pérez-Rodriguez, P., & de los Campos, G. (2017). Bayesian genomic prediction with genotypex environment interaction kernel models. G3: Genes, Genomes, Genetics, 7(1), 41-53
Gauch, H. G., & Zobel, R. W. (1997). Identifying mega-environments and targeting genotypes. Crop Science, 37, 311-326
Granato, I., Cuevas, J., Luna-Vázquez, F., Crossa, J., Montesinos-López, O., Burgueño, J., & Fritsche-Neto, R. (2018). BGGE: A new package for genomic-enabled prediction incorporating genotype x environment interaction models. G3: Genes, Genomes, Genetics, 8(9), 3039-3047
Hayes, B. J., Bowman, P. J., Chamberlain, A. J., & Goddard, M. E. (2009). Invited review: Genomic selection in dairy cattle: Progress and challenges. Journal of dairy science, 92(2), 433-443
Hayes, B. J., Daetwyler, H. D., & Goddard, M. E. (2016). Models for genomex environment interaction: Examples in livestock. Crop Science, 56(5), 2251-2259
He, S., Reif, J. C., Korzun, V., Bothe, R., Ebmeyer, E., & Jiang, Y. (2017). Genome-wide mapping and prediction suggests presence of local epistasis in a vast elite winter wheat populations adapted to Central Europe. Theoretical and Applied Genetics, 130(4), 635-647
He, S., Thistlethwaite, R., Forrest, K., Shi, F., Hayden, M. J., Trethowan, R., & Daetwyler, H. D. (2019). Extension of a haplotype-based genomic prediction model to manage multi-environment wheat data using environmental covariates. Theoretical and Applied Genetics, 132(11), 3143-3154
Gauch Jr, H. G., Piepho, H. P., & Annicchiarico, P. (2008). Statistical analysis of yield trials by AMMI and GGE: Further considerations. Crop science, 48(3), 866-889
Jarquín, D., Crossa, J., Lacaze, X., Du Cheyron, P Daucourt, J., Lorgeou, J., et al. (2014). A reaction norm model for genomic selection using high-dimensional genomic and environmental data. Theoretical and applied genetics, 127(3), 595-607
Jarquin, D., Lemes da Silva, C., Gaynor, R. C., Poland, J., Fritz, A., Howard, R., et al. (2017). Increasing genomic-enabled prediction accuracy by modeling genotypex environment interactions in Kansas wheat. The plant genome, 10(2), 1-15
Jiang, Y., Schmidt, R. H., & Reif, J. C. (2018). Haplotype-based genome-wide prediction models exploit local epistatic interactions among markers. G3: Genes, Genomes, Genetics, 8(5), 1687-1699
Lee, S. H., DeCandia, T. R., Ripke, S., Yang, J., Sullivan, P. F., Goddard, M. E., et al. (2012). Estimating the proportion of variation in susceptibility to schizophrenia captured by common SNPs. Nature genetics, 44(3), 247-250
Lee, S. H., & Van der Werf, J. H. (2016). MTG2: an efficient algorithm for multivariate linear mixed model analysis based on genomic information. Bioinformatics, 32(9), 1420-1422
Lopez-Cruz, M., Crossa, J., Bonnett, D., Dreisigacker, S., Poland, J., Jannink, J. L., et al. (2015). Increased prediction accuracy in wheat breeding trials using a marker×environment interaction genomic selection model. G3: Genes, Genomes, Genetics, 5(4), 569-582
Lourenco, D. A. L., Tsuruta, S., Fragomeni, B. O., Masuda, Y., Aguilar, I., Legarra, A., et al. (2015). Genetic evaluation using single-step genomic best linear unbiased predictor in American Angus. Journal of animal science, 93(6), 2653-2662
Meuwissen, T. H. E., Hayes B. J., & Goddard M. E. (2001). Prediction of total genetic value using genome-wide dense marker maps. Genetics, 157, 1819-1829
Montesinos-López, O. A., Montesinos-López, A., Crossa, J., Montesinos-Lopez, J. C., Luna-Vázquez, F. J., Salinas-Ruiz, J., et al. (2017). A variational Bayes genomic-enabled prediction model with genotype×environment interaction. G3: Genes, Genomes, Genetics, 7(6), 1833-1853
Montesinos-López, O. A., Montesinos-López, A., Crossa, J., Gianola, D., Hernández-Suárez, C. M., & Martin-Vallejo, J. (2018). Multi-trait, multi-environment deep learning modeling for genomic-enabled prediction of plant traits. G3: Genes, Genomes, Genetics, 8(12), 3829-3840
Pérez, P., & de Los Campos, G. (2014). Genome-wide regression and prediction with the BGLR statistical package. Genetics, 198(2), 483-495
Preda, C., Saporta, G., & Mbarek, M. H. (2010). The NI PALS algorithm for missing functional data. Revue roumaine de mathematiques pures et appliquées, 55(4), 315-326
Sattari, M. T., Rezazadeh-Joudi, A., & Kusiak, A. (2017). Assessment of different methods for estimation of missing data in precipitation studies. Hydrology Research, 48(4), 1032-1044
VanRaden, P. M. (2008). Efficient methods to compute genomic predictions. Journal of dairy science, 91(11), 4414-4423
Yan, W., Hunt, L. A., Sheng, Q., & Szlavnics, Z. (2000). Cultivar evaluation and mega-environment investigation based on the GGE biplot. Crop Science, 40(3), 597-605
Yan, W. (2001). GGEbiplot—A Windows application for graphical analysis of multi-environment trial data and other types of two-way data. Agronomy journal, 93(5), 1111-1118
Yan, W., & Kang, M. S. (2002). GGE biplot analysis: A graphical tool for breeders, geneticists, and agronomists. CRC press, Boca Raton, FL
Yang, R -C., Crossa, J., Cornelius, P. L., & Burgueno, J. (2009). Biplot analysis of genotype×environment interaction: Proceed with caution. Crop Science, 49(5), 1564-1576

1-17. (canceled)

18. A method for determining phenotypic genomic estimated breeding values (pGEBVs), wherein the method comprises the steps of:
a) obtaining genetic, phenotypic, and environmental data for a population of organism genotypes;
b) dividing the data of step (a) into a reference population and a validation population; and
c) analysing the data obtained,
wherein the analysis includes:
i. calculating the genotype plus genotype×environment (GGE) principle component (PC) for the data of the reference population;
ii. identifying polymorphisms in the genetic data of the reference population and calculating the polymorphism effect for each PC;
iii. calculating a genomic estimated breeding value (GEBV) for each genotype of the validation population using the calculated polymorphism effect for each PC; and
iv. converting each GEBV into a phenotypic GEBV (pGEBV) by multiplying the GEBV with an inverse of a rotation matrix, wherein the rotation matrix is (e×e), and wherein e is the number of environments in the validation population.

19. The method according to claim 18, wherein the GEBV is a G matrix (n×e), wherein n is the number of validation individuals and e is the number of environments in the validation population.

20. The method according to claim 18, wherein calculating each pGEBV is based on Equation 3 as follows:
pGEBV=G×R⁻¹
where G is an (n×e) matrix of GEBVs for the GGE PCs scaled by multiplying each PC with its standard deviation; n is the number of validation individuals; e is the number of environments; and R⁻¹ is an inverse of the rotation matrix (e×e) or an environment coordinate matrix scaled by dividing each column on a standard deviation of the correspondence PC.

21. The method according to claim 18, wherein the data obtained for the population of organism genotypes is from a plurality of mega-environments.

22. The method according to claim 18, wherein the polymorphisms include a single nucleotide polymorphism.

23. The method according to claim 18, wherein calculating the polymorphism effect for each PC utilises a Bayesian Ridge Regression model.

24. The method according to claim 18, wherein the organism is a plant.

25. The method according to claim 24, wherein the phenotypic data includes records on yield.

26. The method according to claim 25, wherein the environmental data includes irrigation and/or rain exposure.

27. The method according to claim 18, wherein the environmental data includes irrigation and/or rain exposure.

28. The method according to claim 18, wherein the method is used for selecting a genotype for producing an improved organism in a given environment, by one or more of the following:
a. identifying a genotype with the pGEBV which correlates highest with a given environment;
b. clustering the reference environments into mega environments and then calculating multiple averages of pGEBVs per genotype for each mega environment, and identifying a genotype from the average pGEBV that correlates highest with the mega environment which best matches the given environment; and
c. identifying a genotype from the following steps:
i. calculating a singular value decomposition for a symmetric pairwise correlation matrix (U matrix) between environments including the reference environments and the selected environment (e+1×e+1);
ii. calculating a correlation between the U matrix obtained in step (i), and the rotation matrix (e×e);
iii. reordering columns of the U matrix to match an order of the rotation matrix of step (ii), reversing the sign of negative correlations; and
iv. applying Equation 3 as follows:
pGEBV=G×R⁻¹
using the reordered U matrix instead of the R matrix, where G is an (n×e) matrix of GEBVs for the GGE PCs scaled by multiplying each PC with its standard deviation; n is the number of validation individuals; and e is the number of environments; and adding a column of zeros to the end of the G matrix to match its dimensions; and
d. based thereon, selecting an identified genotype.

29. A method according to claim 28, further comprising locating the selected genotype to said given environment.

30. A method for selecting a genotype for producing an improved organism in a given environment, wherein the method comprises the steps of:
e. performing a method for determining phenotypic genomic estimated breeding values (pGEBV) according to claim 18; and performing one or more of:
i. identifying a genotype with a pGEB V which correlates highest with the given environment;
ii. clustering the reference environments into mega environments and then calculating multiple averages of pGEBVs per genotype for each mega environment, and identifying a genotype from the average pGEBV that correlates highest with the mega environment which best matches the given environment; and
iii. identifying a genotype from the following steps:
1. calculating a singular value decomposition for a symmetric pairwise correlation matrix (U matrix) between environments including the reference environments and the given environment (e+1×e+1);
2. calculating a correlation between the U matrix obtained in step 1, and the rotation matrix (e×e);
3. reordering the columns of the U matrix to match an order of the rotation matrix (of step 2, reversing the sign of negative correlations, and applying Equation 3 as follows:
pGEBV=G×R⁻¹
using the reordered U matrix instead of the R matrix, where G is an (n×e) matrix of GEBVs for the GGE PCs scaled by multiplying each PC with its standard deviation; n is the number of validation individuals; and e is the number of environments; and adding a column of zeros to the end of the G matrix to match its dimensions; and
f. based thereon, selecting an identified genotype.

31. The method according to claim 30, wherein reordering columns of the U matrix includes ordering the column of the U matrix with the highest absolute correlation coefficient value with a first column of the rotation matrix (e×e).

32. The method according to claim 30, wherein the given environment is a new environment not included in the reference population.

33. The method according to claim 30, further comprising locating the selected genotype to said given environment.

34. The method according to claim 30, wherein the organism is a plant.

35. The method according to claim 30, wherein the step of calculating a singular value decomposition for a symmetric pairwise correlation matrix between environments uses a non-linear iterative partial least squares (NIPALS) algorithm to approximate missing correlation coefficients.

36. A method for producing an improved organism, comprising the steps of:
g. performing a method for determining phenotypic genomic estimated breeding values (pGEBV) according to claim 18;
h. performing a method for selecting a genotype for producing an improved organism according to claim 30; and
i. locating the organism comprising said selected genotype in said given environment.
